(19) United States
(12) Patent Application Publication
(10) Pub. No.: US 2006/0129141 A1
(43) Pub. Date: Jun. 15, 2006

(54) Treatment of eye disorders using articulated-arm coupled ultraviolet lasers

(76) Inventor: J. T. Lin, Oviedo, FL (US)

Correspondence Address: J. T. LIN, 4532 Old Carriage Trail, Oviedo, FL, 32765, US

(21) Appl. No.: 11/008,108

(22) Filed: Dec. 10, 2004

(51) Int. Cl. A61F 9/008; A61B 18/18

(52) U.S. Cl. 606/5; 606/19; 606/11

(57) ABSTRACT

Surgical method and apparatus for presbyopia correction and glaucoma by laser removal a portion of the sclera and/or ciliary tissue are disclosed. The disclosed preferred embodiments of the system consists of a beam spot controller, an articulated arm and an attached end-piece. The basic laser beam includes UV laser having wavelength ranges of (0.19-0.36) microns, generated from UV excimer lasers of ArF, XeCl or solid state lasers of Nd:YLF, Nd:YAG, Ti:sapphire with harmonic generation using nonlinear crystals. Presbyopia is treated by ablation of the treated surface tissue in predetermined patterns outside the limbus to increase the accommodation of the eye. Glaucoma is treated by decreasing of intra ocular pressure of the laser surgery.

### RELATED APPLICATION

This is a continuation-in-part of Ser. No. 645,569, Aug. 22, 2003, which is now abandoned.

### BACKGROUND OF THE INVENTION

1. Field of the Invention

The present invention relates to methods and system for the treatment of presbyopia and glaucoma using articulated-arm-coupled ultraviolet laser to ablate the sclera or ciliary tissue.

2. Prior Art

Corneal reshaping including a procedure called photorefractive keratectomy (PRK) and a new procedure called laser assisted in situ keratomileusis, or laser intrastroma keratomileusis (LASIK) have been performed by lasers in the ultraviolet (UV) wavelength of (193-213) nm. The commercial UV refractive lasers include ArF excimer laser (at 193 nm) and other non-excimer, solid-state lasers such as those proposed by the present inventor in 1992 (U.S. Pat. No. 5,144,630) and in 1996 (U.S. Pat. No. 5,520,679). The above-described prior arts using lasers to reshape the corneal surface curvature, however, are limited to the corrections of myopia, hyperopia and astigmatism.

Refractive surgery using a scanning device and lasers in the mid-infrared (mid-IR) wavelength was first proposed by the present inventor in U.S. Pat. Nos. 5,144,630 and 5,520,679 and later proposed by Telfair et al. al., in U.S. Pat. No. 5,782,822, where the generation of mid-IR wavelength of (2.5-3.2) microns were disclosed by various methods including: the Er:YAG laser (at 2.94 microns), the Raman-shifted solid state lasers (at 2.7-3.2 microns) and the optical parametric oscillation (OPO) lasers (at 2.7-3.2 microns).

Corneal reshaping may also be performed by laser thermal coagulation currently conducted by a Ho:YAG laser (at about 2 microns in wavelength) proposed by Sand in U.S. Pat. No. 5,484,432. This method, however, was limited to low-diopter hyperopic corrections. Strictly speaking this prior art did not correction the true “presbyopia” and only performed the mono-vision for hyperopic patients. A thermal laser is required and the laser treated area was within the optical zone diameters of about 7 mm.

Ruiz in U.S. Pat. No. 5,533,997 proposed the use of laser ablation of cornea surface to correct presbyopic patients. This prior art, however, must generate multifocal (or bifocal) surface on the central portion of the cornea in order to achieve the desired presbyopia correction. Corneal curvature change by laser ablation in this prior art, however, did not actually resolve the intrinsic problems of presbyopic patient caused by age where the lens loses its accommodation as a result of loss of elasticity due to age.

All the above-described prior arts are using methods to change the cornea surface curvature either by tissue ablation (such as in UV laser) or by thermal shrinkage (such as in Ho:YAG laser) and all are using lasers onto the central potion of the cornea.

The alternative method for presbyopia correction, therefore, is to increase the accommodation of the presbyopic patients by change the intrinsic properties of the sclera and ciliary tissue to increase the lens accommodation without changing the cornea curvature. This method of sclera ablation is fundamentally different from all the prior arts including that of Ruiz, in which reshaping cornea curvature into multifocal shape was required for presbyopia correction.

To treat presbyopic patients, or the reversal of presbyopia, using the concept of expanding the sclera by mechanical devices has been proposed by Schachar in U.S. Pat. Nos. 5,489,299, 5,722,952, 5,465,737 and 5,354,331. These mechanical approaches have the drawbacks of complexity and are time consuming, costly and have potential side effects. To treat presbyopia, the Schachar U.S. Pat. Nos. 5,529,076 and 5,722,952 propose the use of heat or radiation on the corneal epithelium to arrest the growth of the crystalline lens and also propose the use of lasers to ablate portions of the thickness of the sclera. However, these prior arts do not present any details or practical methods or laser parameters for the presbyopic corrections. No clinical studies have been practiced to show the effectiveness of the proposed concepts. The concepts proposed in the Schachar U.S. Pat. Nos. 5,354,331 and 5,489,299, regarding lasers suitable for ablating the sclera tissues were incorrect because he did not identify which lasers are “cold lasers”. Many of his proposed lasers are thermal lasers which will cause thermal burning of the cornea, rather than tissue ablation. Furthermore, the clinical issues, such as locations, patterns and depth of the sclera tissue removal were not indicated in these prior patents. In addition, it is essential to control the desired ablation pattern and to control the ablation depth on the sclera tissue. Schachar's methods also require the weakening of the sclera and increase its diameter by expansion, whereas the proposed concept of the present invention provides new mechanisms for accommodation.

The “presbyopia” correction proposed by Ruitz (U.S. Pat. No. 5,533,997) using an excimer (ArF) laser also required the corneal surface to be reshaped to form “multifocal” effort for a presbyopia patents to see near and far. However, Ruitz's “presbyopia” correction is fundamentally different from that of the present patent which does not change the corneal curvature. The presbyopia correction proposed in the present patent is to increase patient's accommodation rather than reshaping the cornea into “multifocal” surface.

The technique used in the prior art of Bille (U.S. Pat. No. 4,907,586) required a quasi- continuous laser having pulse duration less than 10 picoseconds and focused spot less than 10 micron diameter and the laser is confined to the interior of a selected tissue to correct myopia, hyperopia or astigmatism. Bille also proposed the laser to focus into the lens of an eye to prevent presbyopia. This prior art system is very complicate and needs a precise control of the laser beam size and focusing position. Furthermore, clinical risk of cataract may occur when laser is applied into the lens area.

Another prior art proposed by Spencer Thornton (Chapter 4, “Surgery for hyperopia and presbyopia”, edited by Neal Sher (Williams & Wilkins, MD, 1997) is to use a diamond knife to incise radial cuts around the limbus areas. It requires a deep (90%-98%) cut of the sclera tissue in order to obtain accommodation of the lens. This method, however, involves a lot of bleeding and is difficult to control the depth of the cut which requires extensive surgeon's skill. Another drawback for presbyopia correction provided by the above-described incision methods is the major post-operative regression of about (30%-80%). And this regression is minimum in the laser-ablation method proposed in the present invention. We note that there is intrinsic difference between the ablation-method proposed in this invention and the knife-incision. The sclera space produced by the incision method is not permanent and may be greatly reduced during the tissue healing and cause the regression. This major source of regression in incision method however will not occur in the laser or non-laser ablation-methods as proposed in this invention, where portion of the sclera tissue is permanently removed.

One of the important concepts proposed in the present invention is to support the post-operative results which show minimum regression. We proposed that the laser ablated sclera tissue “gap” will be filled in by the sub-conjunctival tissue within few days after the surgery. This filled in sub-conjunctival tissue is much more flexible than the original sclera tissue. Therefore the filled-in gap in the sclera area will cause the under laying ciliary body to have more space to move. This in turn will allow the ciliary body to contract or expand the zonular fiber which is connected to the lens, when the presbyopic patient is adjusting his lens curvature to see near and far. The above described sub-conjunctival tissue filling effects and the increase of “flexibility” of the sclera area are fundamentally different from the scleral “expansion” (or weakening) concept proposed by the prior arts of Schachar and proposed by the implant of a scleral band. In the present invention, the laser ablated sclera area is not weakening, it becomes more flexible instead.

The prior art by the present inventor, U.S. Pat. No. 6,263,879 was limited to scanning device which has drawback of de-centration caused by eye movement, and it is hard to control the ablation depth due to the fact that the scanning laser beam is not perpendicular to the scleral surface. Another prior art of Lin, U.S. Pat. No. 6,258,082 proposed the fiber-coupled lasers which however was mainly designed for an IR-laser because the coupling efficiency of existing fibers was very poor, less than 30%, when laser wavelength shorter than 0.27 microns. There was no disclosure of specific designs for articulated arm. Furthermore, the above prior art is limited to laser ablation depth (400 microns) or about 80% of the scleral layer, which suffers post-surgery regression. Much deeper ablation depth up to 1200 microns is proposed in the present invention in order to achieve better outcome and minimal regression.

In addition, the fiber-coupled laser has drawbacks of high cost and can be easily damaged, particularly the fiber tip which is contacted to the scleral tissue. No high UV-transparent fibers are currently available for the application of high peak-power UV lasers, particularly for the spectral range of (0.19-0.3) microns, operated in the nanosecond pulse duration.

Articulated arms have been commercially used to deliver laser beams. However, they are mainly used for dermatological uses and are limited to spectrum of visible (500-700) nm and IR at (1-3) microns or at about 10.6 micron. No UV lasers of (190-300) nm have been developed and coupled to articulated arm for the treatment of eye disorders including presbyopia and glaucoma. Furthermore, the articulated arm for prior art uses did not require a good beam alignment or centration, because of a rather large beam spot (4-15) mm, are normally used. On the contrary, the presently proposed UV laser, articulated-arm system requires centration/alignment better than 0.2 mm because of its much smaller beam spot of (0.2-1.0) mm needed in the present invention. The dermatological lasers are using the laser thermal effects to change the skin conditions, whereas the present invention requires a “cold” laser for tissue ablation and thermal effects must be minimized.

Precise centration or beam directional stability (better than 0.2 mm) and beam spot size control (better than 0.15 mm) are the two key features required for the present by proposed laser system and procedures. Systems with these features have not been previously disclosed or designed due to design difficulty and lack of commercial needs in the existing laser procedures other than the vision correction presented in the present invention.

One objective of the present invention is to provide an apparatus and method to obviate these drawbacks in the prior arts.

It is yet another objective of the present invention to disclose a specific articulated arm with arm configuration and mirror mounting means designed to meet the above described specific features which are required in the proposed procedures.

It is yet another objective of the present invention to use an articulated-arm-coupled lasers such that the accommodation of a presbyopic eye can be increased by specific ablation patterns, location, size and shapes of the removed tissue outside the limbus of the eye.

It is yet another objective of the present invention to define the non-thermal lasers for efficient tissue ablation to prevent refractive power change of the cornea caused by thermal effects.

It is yet another objective of the present invention to define the optimal laser parameters and the ablation patterns for best clinical outcome for presbyopia patients, where sclera and/or ciliary ablation (with much deeper ablation depth) will increase the accommodation of the ciliary muscle by the increase of the flexibility in the laser-ablated areas.

It is yet another objective of the present invention to provide the appropriate ablation patterns which will cause effective ciliary body contraction and expansion on the zonules and the lens.

It is yet another objective of the present invention to provide a new mechanism which improves the clinical results of presbyopia correction with minimum regression.

It is yet another objective of the present invention to provide an articulated-arm device to achieve the coupling efficiency required in the proposed procedures.

The present invention described in great detail for the treatment of presbyopia may be extended to other eye disorders including glaucoma. For the case of glaucoma, the laser may be used to remove sclera tissue in the area where Schlemm's channel is located followed by a removal of a small portion of the iris underlying this area.

The invention having now been fully described, it should be understood that it may be embodied in other specific forms or variations without departing from the spirit or essential characteristics of the present invention. Accordingly, the embodiments described herein are to be considered to be illustrative and not restrictive.

### SUMMARY OF THE INVENTION

The preferred embodiments of the basic surgical lasers of the present invention shall include ultraviolet (UV) lasers having wavelength range of about (190-360) nm, such as ArF (at 193 nm) and XeCl (at 308 nm) excimer lasers, nitrogen laser (at 337 nm) flash-lamp-pumped and diode-pumped solid state lasers having wavelength range of about (190-355) nm such as Nd-YAF, Er:YAG, Nd:YAG, Er:glass and Ti:saphire laser using harmonic generation from nonlinear crystals of KTP, BBO, LBO, KDP and other UV transparent crystals.

It is yet another preferred embodiment is to couple the basic lasers by an articulated-arm to deliver the laser beam to treated area of the eye, in which the end of the arm is connected to a short tip-tube which may be disconnected for reuse after sterilization.

It is yet another preferred embodiment to focus the laser beams into a desired spot size on the treated area of the eye. Various ablation patterns may be generated manually via the hand piece including multiple rings of spots, radial line or non-specific shapes outside the limbus.

It is yet another preferred embodiment to control laser beam spot size by position and focal length of focusing lens and the length of the attached end pieces.

It is yet another preferred embodiment to control laser beam centration by means of reflecting mirrors which are mounted on the joints of the arm and each mount can be angle fine-tuned independently.

It is yet another preferred embodiment is to ablate by the basic UV lasers, a portion of the sclera and ciliary tissue to increase the flexibility and available space of the sclera-ciliary-zonus complex to increase the lens accommodation (for presbyopia) and reduce the intraocular pressure (IOP) of the eye (for glaucoma treatment).

It is yet another preferred embodiment to prepare a flap of the conjunctiva layer prior to the laser ablation of the under-layer of the sclera tissue for a better control of the ablation depth and for safety reasons.

It is yet another preferred embodiment is that the ciliary body may also be ablated by the UV laser with or without ablating the conjunctival or scleral layer.

Further preferred embodiments of the present invention will become apparent from the description of the invention which follows.

### BRIEF DESCRIPTION OF THE DRAWINGS

FIG. 1. System schematics shows the laser output coupled to an articulated arm.

FIG. 2. Structure of the mounted mirrors for angle tuning.

FIG. 3. Structure of focusing optics and end piece of the articulated arm.

### DETAILED DESCRIPTION OF THE INVENTION AND THE PREFERRED EMBODIMENTS

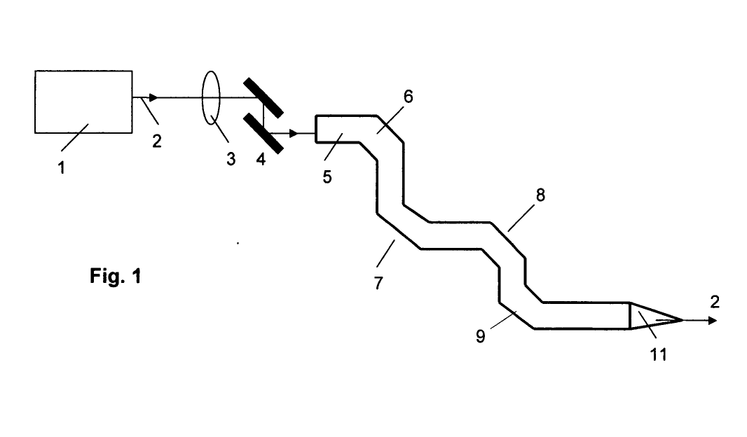

A surgical laser system in accordance with the present invention (as shown in FIG. 1) comprises a basic laser 1 having wavelength in the UV spectrum 2 is focused by lens 3 and coupled by a pair of reflecting mirrors 4 to an articulated-arm (ATA) 5 which uses a set of UV high reflecting coated mirrors mounted to each of the joints 6-9, and has an end piece 11. These mounts are independently adjustable for fine-tuning of laser alignment and centration in order to meet our required beam directional stability (smaller than 0.2 mm) and spot size accuracy (better than 0.15 mm).

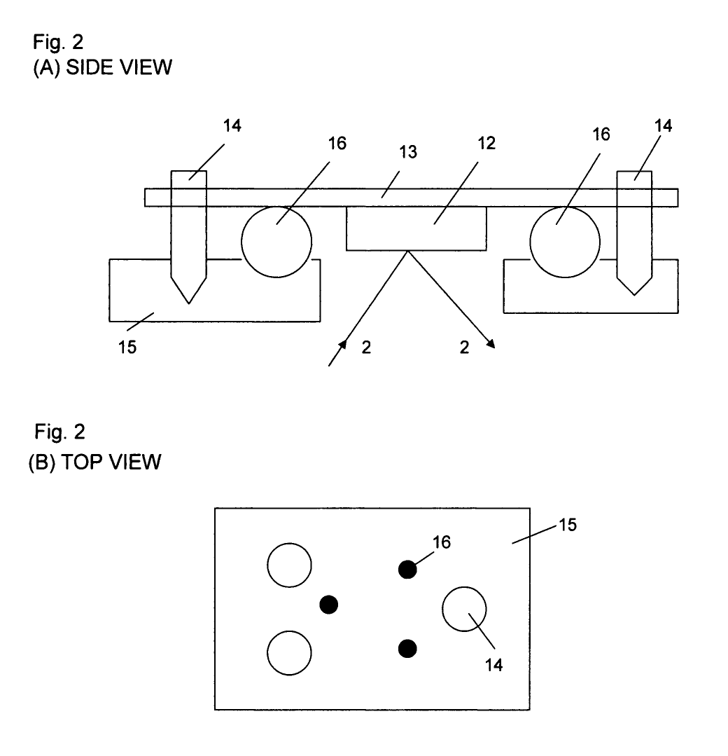

As shown in FIG. 2A (the side view of the mount), the UV reflecting mirror 12 is mounted to a base 13 which can be angled tuned to change the reflection angle (approximately 45 degree) of the incident laser beam 2 by three screws 14 attached to the joint body of the articulated arm 15 which has three supporting metal balls 16 glued to the joint body 15. FIG. 2B shows the top view of the design, where the base 13 and mounted mirror 12 can be fined tuned by the three screws 14 against the metal balls 16 to change the direction of the incident laser 2 (FIG. 2A). The preferred dimensions of the components in FIG. 2 are: diameter of (2-3) mm for the metal ball, length of (4-5) mm for the screws 14, (0.5-1.5) mm thickness of the reflecting mirror 12 which is about 5×5 mm square in area and has front surface coated to reflect UV laser at an angle approximately 45 degree. Another preferred feature is that each of the mirrors mounted to the joints can be adjusted independently for best centration and directional stability which are critical in the present invention. Another preferred embodiment is to use a visible laser such as HeNe or red diode laser to align the incident beam which is reflected from the mirrors mounted to each of the joint. After centration is achieved within 0.2 mm stability at the output end of the articulated arm while the arm is rotated in a wide angle of (20-45) degree in 3-dimension, the screws 14 are glued to the base 13 to avoid mechanical movement of their fine tuned angles.

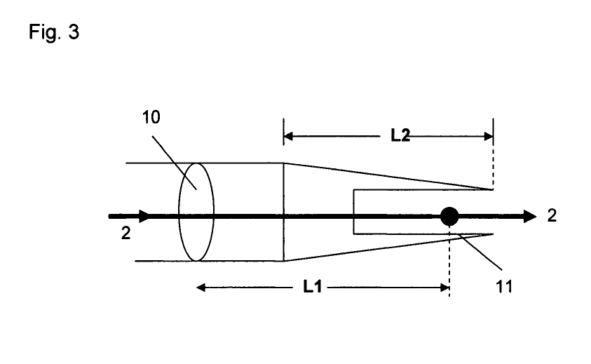

As shown in FIG. 3, the yet another preferred embodiment is that the end piece 11 contacts the treated eye surface such that laser beam spot size and its location are well defined by the focal length of the optics 10 and the length of the end piece 11. Using high reflecting UV mirrors, we are able to achieve an overall coupling efficiency over 75% when an articulated-arm having 4 joints is used. This efficiency is much higher than when a fiber is used which is less than 30% for spectrum range of (195-280) nm. As shown in FIG. 3, the laser beam 2 is focused by a lens 10 having a preferred focal length of (10-15) cm shown by L1, such that the beam is focused at a position less than the length of the attached end piece 11 having a length of L2=(5-10) cm. Specific of the relative length of L1 and L2 gives controlled beam spot size and a divergent beam for safety when the laser beam ablates the eye tissue.

The proposed UV laser provides a “clean” cut with almost no thermal tissue damage, whereas prior art using an IR laser (LIN, U.S. Pat. No. 6,258,082) at about 2.9 microns suffers certain degree of thermal damage, particularly around the laser spot edge which has less power. It is critical that thermal effects on the cornea must be minimized, otherwise patient's far vision will become “hyperopic shift” caused by the thermal shrinkage of the cornea. This drawback of prior art using IR laser can be eliminated when the proposed UV laser is used. Prior arts using a fiber and a fiber tip to deliver the laser energy also suffers fiber-tip damage when laser heating is accumulated at the tip end. IR fiber tip end can be easily stuck with sclera tissue and cause available laser power to drop significantly. All these prior art drawbacks are obviated in the proposed UV laser system coupled to an articulated arm which also has a much higher coupling efficiency than that of an IR laser.

The preferred articulated arm shall have a length about (0.5-1.2) meter, a minimum of 2 joints (for free rotation in x, y and z directions), connected to an end piece with length about (5-10) mm. In addition, at least 2 highly UV reflecting mirrors are mounted at the joint position to reflect the laser beam along the arm tube with a centration better than 0.2 mm. The preferred laser spot diameter is about (0.1-1.0) mm with UV energy per pulse of about (0.5-10) mJ on the treated surface, or at the output end of the articulated arm. It is also a preferred requirement that the laser output alignment from the arm should not deviate more than 0.2 mm while keeping its output energy stability better than 10%, when the arm is freely rotated in 3 dimension.

According to the present invention, the preferred embodiments of the basic surgical lasers for presbyopia correction and/or glaucoma procedures shall include: (a) ultraviolet (UV) lasers having wavelength range of about (190-360) nm, such as ArF (at 193 nm) and XeCl (at 308 nm) excimer lasers, nitrogen laser (at 337 nm); and (b) solid-state lasers using harmonic generation from solid-state lasers of Nd:YAG, Nd:YLF and Alexandrite lasers where nonlinear crystals of KTP, BBO or KDP may be used to up convert the fundamental frequency to the desired UV (0.19-0.3) microns range. These solid-state lasers may be flash lamp pumped or diode laser pumped.

According to one aspect of the present invention, the preferable UV laser energy per pulse on the treated eye surface is about (0.5-10) mJ. Focused spot size of about (0.1-1.0) mm in diameter on the treated eye surface is achieved by means of focusing which consists of at least one spherical lens with focal length of about (5-100) cm. The other preferred laser parameter of this invention is the laser repetition rate range of about (5-100) Hz which will provide reasonable surgical speed and minimum thermal effects. The focused beam delivered to the articulated arm may be scanned over the treated eye surface for various patterns by surgeon's control of the hand piece or by a commercially available scanner attached to the end piece.

Another preferred embodiment is to use a cylinder focusing lens to generate slit shape size of (0.1-0.5)×(3-5) mm.

The preferred patterns of this invention include a ring-spot having at least one ring with at least 3 spots in each ring, a radial-pattern having at least 3 radials or non-specific shapes as far as it is in a symmetric form. The preferred area of the ablation is defined within two circles having diameters about 10 mm and 14 mm posterior to the limbus along the radial direction of the cornea. We should note that for the case of a circular laser spot, a radial ablation pattern on the treated eye surface may be generated either by manually or motorized scan. For the situation of the slit spot, the surgeon may easily generate the radial patterns without moving the end tip of the articulated arm.

The preferred ablation depth of the ablated tissue is about (400-1200) microns and most preferable about (600-1200) microns with each of the radial length of about (1.0-5.0) mm adjustable according to the optimal clinical outcomes including minimum regression and maximum accommodation for the presbyopic patients. We note that the ablation depth of up to 600 microns or limited to about 80% of the scleral tissues in the prior art of Lin (U.S. Pat. No. 6,258,082) suffers post-surgery regression. The much deeper ablation depth (up to 1.2 mm), about (10%-50%) deep into the ciliary body, proposed in this invention will reduce the regression and also improve the clinical outcome. The preferred radial ablation shall start at a distance about (4.0-5.5) mm from the corneal center and extended about (2.0-5.0) mm outside the limbus. The preferred embodiments of the radial patterns on the sclera area include at least 3 radial lines, curved lines, ring-dots or any non-specific shapes as far as they are symmetric to the center of the eye. The symmetric form is required to achieve an even “force” acting on the zonus fiber for lens relaxation. Any other non-specific patterns including curved lines, z-shape, t-shape lines around the area outside the limbus should be within the scope of this patent.

It is yet another preferred embodiment is to control the laser spot on the treated surface by positioning of the focusing lens at about (0.5-1.0) focal length away from the output end of the articulated arm. This focal lens is preferred to be integrated inside the last section (last joint) of the arm having a typical length about (5-20) cm. For safety issue, the preferred laser beam is focused (0.5-1.5) cm above the treated surface such that the beam is divergent when it is delivered to the treated surface. When surgeon's hand piece is held away from the eye surface, the laser beam spot is always divergent and expanding to a low power level. High power ablating spot is available only when end-top of the articulated arm is contacted to the eye surface.

It is yet another preferred embodiment is to ablate a portion of the treated eye surface for the treatment of eye disorders including prebyopia, (by increasing accommodation) and glaucoma (by reducing the intraocular pressure). In each procedure, the sclera-ciliary complex becomes more flexible with increasing space between the ciliary ring and the lens.

It is yet another preferred embodiment is to ablate a portion of the treated eye surface which includes removal of portion of (about 10% to 50% in depth) the ciliary body with or without the removal of the conjunctival and scleral layers, or removal of portion of the scleral layer (about 80%) without removing the ciliary body.

While the invention has been shown and described with reference to the preferred embodiments thereof, it will be understood by those skilled in the art that the foregoing and other changes and variations in form and detail may be made therein without departing from the spirit, scope and teaching of the invention. Accordingly, threshold and apparatus, the ophthalmic applications herein disclosed are to be considered merely as illustrative and the invention is to be limited only as set forth in the claims.

I claim:

1. A surgical method for treating eye disorder of presbyopia and glaucoma by removing a portion of the surface tissue of an eye comprising the steps of:
(a) selecting a laser beam having a predetermined energy, spot size and wavelength;
(b) selecting a beam spot controller mechanism to focus said laser beam to an articulated arm;
(c) controlling said articulated arm to deliver said laser beam in a predetermined pattern onto a plurality of positions on the eye to remove a portion of said surface tissue outside the limbus area;
whereby the treated eye will have increased vision accommodation and decreased intra ocular pressure.

2. A surgical method of claim 1, wherein said laser beam is an ultraviolet laser having a wavelength range of about (0.19-0.36) microns and a pulse energy of about (0.5-10) mJ on said surface tissue.

3. A surgical method of claim 1, wherein said laser beam is an excimer laser having a wavelength of 193 nm or 308 nm.

4. A surgical method of claim 1, wherein said beam spot controller consists of at least one spherical focusing lens to couple said laser beam to said articulated arm.

5. A surgical method of claim 1, wherein said articulated arm consists of at least 2 joints mounted with highly ultraviolet reflecting mirrors which can be angle tuned independently for a centration accuracy better than 0.2 mm.

6. The surgical method of claim 5, wherein said centration accuracy is achieved by three screws connecting the base of the mounted mirror and the joint body of said articulated arm.

7. The surgical method of claim 5, wherein said articulated arm is further connected to an end piece having a length about (5-10) cm such that said laser beam is divergent at said surface tissue.

8. The surgical method of claim 5, wherein said articulated arm is able to coupled at least 75% of the input said laser beam energy to said surface tissue with a spot size of (0.1-1.0) mm.

9. The surgical method of claim 1, wherein said surface tissue is ablated by said laser beam to a depth of about (400-1200) microns.

10. A surgical method of claim 1, wherein said predetermined pattern includes radial lines, curved lines, ring-dot or non-specific patterns around the area outside the limbus.

11. A surgical method of claim 1, wherein said surface tissue includes the conjunctiva layer, sclera tissue and ciliary body.

12. A surgical method of claim 1, wherein removing a portion of the said surface tissue includes removal of about 80% of the scleral thickness with or without removal of the conjunctival layer.

13. A surgical method of claim 1, wherein removing a portion of the said surface tissue includes removal of about (10%-50%) of the diary body depth with or without removal of the conjunctival or scleral tissue.

14. A system for treating presbyopia and glaucoma, the system comprising
(a) a laser beam having a predetermined energy, spot size and wavelength;
(b) a beam spot controller mechanism to focus said laser beam to an articulated arm;
(c) controlling said articulated arm to deliver said laser beam in a predetermined pattern onto a plurality of positions on the eye to remove a portion of said surface tissue outside the limbus area;
whereby the treated eye will have increased vision accommodation and decreased intra ocular pressure.

15. A system of claim 14, wherein said laser beam is an ultraviolet laser having a wavelength range of about (0.19-0.36) microns and a pulse energy of about (0.5-10) mJ on said surface tissue.

16. A system of claim 14, wherein said articulated arm consists of at least 2 joints mounted with highly ultraviolet reflecting mirrors which can be angle tuned independently for a centration accuracy better than 0.2 mm.

17. A system of claim 14, wherein said centration accuracy is achieved by three screws connecting the base of the mounted mirror and the joint body of said articulated arm.

18. A system of claim 14, wherein said surface tissue is ablated by said laser beam to a depth of about (400-1200) microns.

19. A system of claim 14, wherein removing a portion of the said surface tissue includes removal of about (10%-50%) of the ciliary body depth with or without removal of the conjunctival or scleral tissue.
